(19) United States
(12) Patent Application Publication
(10) Pub. No.: US 2023/0332091 A1
(43) Pub. Date: Oct. 19, 2023

(54) BIOREACTOR SYSTEMS AND METHOD FOR OPERATING A BIOPROCESS

(71) Applicant: Sartorius Stedim Biotech GmbH, Göttingen (DE)

(72) Inventors: Marco Leupold, Göttingen (DE); Simon Topp-Manske, Lohfelden (DE); Ute Husemann, Göttingen (DE); Sebastian Ruhl, Fulda (DE); Sascha Gärtner, Sontra (DE); Yannick Stapel, Göttingen (DE); Regina Wollschmidt, Uslar (DE); Gerhard Greller, Göttingen (DE); Thomas Dreher, Göttingen (DE)

(73) Assignee: Sartorius Stedim Biotech GmbH, Göttingen (DE)

(21) Appl. No.: 18/044,641

(22) PCT Filed: Jun. 17, 2021
(86) PCT No.: PCT/EP2021/066333

§ 371 Date: Mar. 9, 2023

(30) Foreign Application Priority Data
Sep. 10, 2020 (DE) 10 2020 005 550.8

(51) Int. Cl. C12M 1/34; C12M 1/00; C12M 1/06; C12M 3/00

(52) U.S. Cl. CPC C12M 41/42; C12M 23/28; C12M 23/14; C12M 27/02; C12M 23/22; C12M 23/48; C12M 41/12; C12M 27/20

(57) ABSTRACT

A bioreactor system for receiving a disposable bioreactor bag comprises a receiving container having a container wall which defines a receiving space in which the disposable bioreactor bag is received in an operating state of the bioreactor system. A stirring system projects at least partially into the receiving space and is designed and configured to stir a biomedium present in the disposable bioreactor bag in the operating state of the bioreactor system. At least one baffle, which makes the receiving space smaller and differs from the container wall, serves to reduce a laminar flow of the biomedium. A temperature control medium flows through at least part of the at least one baffle, said temperature control medium controlling the temperature of the baffle.

### TECHNICAL FIELD

The invention relates to bioreactor systems for receiving a disposable bioreactor bag as well as methods for operating a bioprocess.

### BACKGROUND

Bioreactor systems are used for receiving, storing, and cultivating biological media such as fluids. The biomedium can be provided in a disposable bioreactor bag, which can have a volume of a few liters up to several hundred liters. The disposable bioreactor bag with the biomedium is inserted into the bioreactor system, in which the biomedium is heated to a predeterminable temperature over a predetermined period of time, usually several hours. Furthermore, in such a bioreactor system, various investigations can be carried out on the biological medium.

A bioreactor can be handled under clean room conditions, so that particularly high demands are placed on the quality assurance of the bioreactor. In particular, high quality demands are placed on the temperature control and mixing of the biological medium.

A bioreactor system for the cultivation of animal cells is known from the publication WO 2016/192824 A1. Bioreactor systems for some intensified cell culture processes, such as microbial processes, phototrophic processes, and processes with fungal cells, still pose technical problems. Cultivation of such cells can require increased oxygenation, more intensive mixing (i.e. increased stirring speed and/or stirring power) and/or an improved cooling. Each bioprocess can (e.g. depending on the cells to be cultivated) place individual demands and/or functions on the bioreactor system. In comparison to cell culture processes, microbial processes require an oxygen transfer that is several times higher and a cooling rate that is several times higher. The culture broth in fungal processes is often very viscous, for which reason a suitable bioreactor system should meet special demands in terms of power input and stirring efficiency.

### SUMMARY

The problem addressed by the invention is to make it possible to carry out a microbial bioprocess and/or phototrophic bioprocess and/or a bioprocess with fungal cells.

This problem is solved by the subject-matter of the independent claims. Preferred embodiments are the subject-matters of the dependent claims.

A first aspect relates to a bioreactor system for receiving a disposable bioreactor bag with a receiving container having a container wall that defines a receiving space in which the disposable bioreactor bag is received when the bioreactor system is in an operating state. A stirring system projects at least partially into the receiving space and is designed and configured to stir a biomedium present in the disposable bioreactor bag in the operating state of the bioreactor system. The bioreactor system comprises at least one baffle, which reduces the size of the receiving space and differs from the container wall, serving to reduce a laminar flow of the biomedium. The at least one baffle is at least partially flowed through by a temperature control medium, which temperature-controls the baffle.

The bioreactor system can be configured to receive a disposable bioreactor bag having a working volume from about 5 liters to about 10,000 liters. The receiving container is designed so as to be robust enough to be used repeatedly in order to carry out a bioprocess. The receiving container is designed as a reusable element of the bioreactor system, similar to at least parts of the stirring system of the bioreactor system, e.g. like a stirring drive. The disposable bioreactor bag can be designed with a flexible plastic bag wall so as to be disposed of after each bioprocess.

The receiving container can be made of stainless steel, for example, in order to enable high stability, sterility, and/or durability. The receiving container comprises the container wall that defines the receiving space. The receiving space can, for example, be substantially cylindrical, e.g. having a convex cylinder bottom and/or cylinder top. Individual elements of the bioreactor system can be configured in a manner similar to the one disclosed in the publication WO 2016/192824 A1 cited above. This applies in particular to the receiving container, the stirring system, doors, and/or cooling systems of the container wall.

The bioreactor system can be configured in particular for intensified cell culture processes of different systematic hierarchies, microbial processes, phototropic processes, and/or processes with fungal cells.

The receiving container wall of the receiving container forms a stable support for the flexible walls of the disposable bioreactor bag during the bioprocess. The disposable bioreactor bag and/or the biomedium can remain arranged inside the receiving space during a large part of and/or during the entire bioprocess. During the bioprocess, parts of the biomedium can be taken, e.g. as samples, and/or ingredients can be added to the biomedium. Ports and/or lines can be formed for this purpose, through which fluids can be supplied and/or discharged. For example, a pressure relief valve for venting gases can be provided, as well as an outlet line and/or an inlet line and/or a circulation line for the biomedium.

The stirring system is used in order to mix the biomedium during the bioprocess. For this purpose, the stirring system can have at least one stirring shaft, which at least partially projects into the receiving space and/or penetrates it completely. Stirring elements and/or at least one stirring and/or mixing element can be arranged on the stirring shaft for thorough mixing of the biomedium during the bioprocess.

In order to at least reduce the occurrence of a laminar flow of the biomedium during stirring, the at least one baffle is formed in the receiving space. When the biomedium is mixed, the baffle can generate turbulence, which disrupts the laminar flow and thus improves the mixing of the biomedium. A plurality of baffles, which can be designed differently, can preferably be arranged in the receiving space.

The baffle can, for example, be arranged adjacent to and/or on an approximately smooth interior of the container wall of the receiving container, in particular on a concave interior (viewed from the interior) of a curved container wall. There, the baffle can break through the smooth inner surface of the container wall in such a way that turbulence occurs during stirring. The larger and/or longer the baffle is, the more and/or stronger turbulence can be generated. However, the baffle can also be arranged spaced apart from the container wall in the receiving space, as long as it is in physical contact with the biomedium and forms a physical and/or irregular barrier in the mixing space upon being mixed. This can already be sufficient in order to reduce the laminar flow.

Because the baffle is different from the container wall, conventional baffles are not temperature-controlled like the container wall. If the baffle is not temperature-controlled, the effective temperature control surface for the biomedium is reduced. At least in the case of conventional baffles, their contact surfaces with the biomedium are not used for temperature control and therefore do not contribute to the cooling capacity.

The temperature-controlled baffle eliminates this disadvantage of non-temperature-controlled baffles in that the temperature control medium can flow through it at least partially. For this purpose, the baffle can have a temperature control channel, for example, through which the temperature control medium can flow. The temperature control medium can preferably flow through the baffle along its entire propagation direction, as a result of which substantially the entire baffle can be temperature-controlled. The baffle can be made of a highly thermally conductive material, such as a metal, in particular stainless steel, in order to enable an effective temperature control of the biomedium by means of good heat conduction.

In particular, a cooling fluid can be used as the temperature control medium, e.g. a similar or the same cooling fluid that is also used in order to cool the container wall. Alternatively, a further temperature control medium can also be used for the at least one baffle, for example air cooling.

In other words, the bioreactor system can have a cooling system, which is used in order to cool the container walls and/or to cool or temperature-control the at least one baffle.

The potential cooling capacity of the bioreactor system is improved by the temperature control of the baffle. Even demanding and/or intensified cell culture processes such as microbial processes and/or processes with fungal cells can be made possible.

The baffle is designed as a mechanical obstacle in the receiving space. The mechanical obstacle is designed and configured so as to influence and/or change the flow behavior of the biomedium when the biomedium is mixed by means of the stirring system. In particular, this can lead to a reduction in the laminar flow, i.e. for example to turbulence that improves and/or intensifies the mixing of the biomedium.

The biomedium can be designed in particular as a liquid biomedium.

According to one embodiment, the at least one baffle comprises at least a first baffle type, which abuts the container wall of the receiving container in such a way that it protrudes from the container wall and projects into the receiving space. This means that the bioreactor system comprises at least one baffle of the first baffle type. The bioreactor system preferably comprises a plurality of baffles of the first baffle type. The baffle of the first baffle type rests against the container wall of the receiving container and can therefore have a baffle side surface facing the container wall. The baffle can, for example, be elongated and can extend along the container wall, in particular from a lower end to an upper end. The baffle can in particular extend in a direction which is arranged approximately parallel to a stirring shaft of the stirring system. By protruding from the container wall, the baffle of the first baffle type has an expansion component which is arranged in an approximately radial direction to the stirring shaft of the stirring system. As a result, the turbulence can be generated for a better mixing of the biomedium.

According to one embodiment, the at least one baffle comprises at least a second baffle type, which extends through the receiving space at least along a section spaced apart from the container wall of the receiving container. The bioreactor system can comprise at least one baffle of the second baffle type, and preferably it comprises a plurality of baffles of the second baffle type. The baffle of the second baffle type can, for example, hang down from above into the receiving space and the disposable bioreactor bag and/or penetrate the receiving space from a first, e.g. upper, end spaced apart from the container wall to a second, e.g. lower, end. The baffle of the second baffle type projects at least partially into the disposable bioreactor bag. This enables a temperature control, in particular a cooling, of the biomedium in a spatial region which is spaced apart from the container wall. This increases the overall cooling capacity that can be transferred to the biomedium, which can enable the processing of more intensive cell cultures.

According to one embodiment, the baffle has a differential temperature control channel, through which the temperature control medium flows through the baffle in two opposite directions. For example, the differential temperature control channel can flow substantially completely through the baffle in the two opposite directions, for example in vertical directions upwards and downwards. The temperature control medium passes through the baffle twice and can release its cold content particularly well. Furthermore, in this case, only one interruption of the container wall is required for introducing and discharging the temperature control medium into and out of the baffle, for example only at an upper end of the baffle. From there, it can first run all the way down through the baffle and from below all the way up again to the upper end of the baffle. This dual conduction of the temperature control medium through the baffle can bring about a particularly uniform cooling at the upper and lower end of the baffle. At the inlet and outlet end of the baffle, the temperature control medium is both the coolest, namely when the temperature control medium is introduced, and the warmest, namely when the temperature control medium is discharged after the biomedium has been temperature-controlled. At the opposite return end in the baffle, the temperature control medium has an approximately medium temperature, because it has already passed through the baffle once. Overall, the cooling performance averages out in such a way that the cooling performance at the inlet and outlet end of the baffle is roughly as strong as at the return end of the baffle. This enables a relatively uniform and therefore controlled cooling of the biomedium.

The differential temperature control channel can be formed both in a baffle of the first baffle type and in a baffle of the second baffle type.

According to one embodiment, at least one cooling bridge is arranged within the baffle on at least one baffle wall, which is abutted by a wall of the disposable bioreactor bag in the operating state of the bioreactor system. The cooling bridge can, for example, be surrounded by the flow of temperature control medium and can project into an interior region of the baffle. The cooling bridge can improve the cooling and can in particular reduce strong temperature fluctuations during the cooling.

According to one embodiment, the baffle penetrates the receiving space approximately completely along an approximately vertical direction. This can apply both to a baffle of the first baffle type and of the second baffle type. In this case, the baffle has an upper end and a lower end, wherein the upper end does not necessarily have to be arranged exactly above the lower end of the baffle; rather, it can be offset laterally thereto. The baffle can be designed so as to be substantially rectilinear and/or have a rectilinear section at least in the interior of the receiving space, along which it penetrates the receiving space substantially completely.

According to one embodiment, the baffle is designed so as to project from one end of the receiving space into the receiving space. For example, it can project from an upper end into the receiving space without being fastened to the container wall at the lower end. The baffle thus has a free end at an end opposite the fixed end of the baffle.

The bioreactor system can have different baffles, for example at least one baffle of the first baffle type and at least one baffle of the second baffle type. The bioreactor system itself can have different baffles of the first baffle type and/or different baffles of the second baffle type.

A second aspect relates to a bioreactor system for receiving a disposable bioreactor bag, which can be designed in particular as a bioreactor system according to the first aspect. The bioreactor system comprises a receiving container having a container wall which defines a receiving space in which the disposable bioreactor bag is received in an operating state of the bioreactor system. A stirring system is configured so as to project at least partially into the receiving space and is designed and configured to stir a biomedium present in the disposable bioreactor bag in the operating state of the bioreactor system. At least one baffle makes the receiving space smaller and differs from the container wall, serving to reduce a laminar flow of the biomedium, which abuts the container wall of the receiving container in such a way that it protrudes from the container wall and projects into the receiving space. The baffle is configured so as to be rounded, such that a wall of the baffle and/or at least a transition from the container wall of the receiving container to the wall of the baffle abutting the former, which wall and/or transition is abutted by disposable bioreactor bag in the operating state, is configured so as to be substantially edgeless.

The bioreactor system according to the second aspect can in particular be an embodiment of the bioreactor system according to the first aspect. Therefore, the description of the bioreactor system according to the first aspect relates at least in part to the bioreactor system according to the second aspect and vice versa. In particular, the bioreactor system according to the second aspect can be a bioreactor system according to the first aspect, wherein the at least one baffle is designed as a baffle according to the first baffle type. This baffle at least partially abuts the container wall of the receiving container. In particular, the baffle can be formed completely along the container wall of the receiving container.

The baffle is rounded in form. The baffle is preferably designed completely without edges, at least on the side and/or the sides abutted by the disposable bioreactor bag in the operating state. Due to the rounded design, air pockets between the disposable bioreactor bag and the baffle and/or the container wall can be reduced. The edgeless shape of the baffle preferably makes it possible for the disposable bioreactor bag to abut the container wall and/or the baffle in the operating state substantially free of air pockets. This reduces air pockets, which can have an insulating effect and thus impede and/or weaken the temperature control of the biomedium. This improves the temperature control and enables a more effective cooling of the biomedium.

In particular, the baffle can be designed in such a way that it does not itself have any sharp edges, rather only rounded edges. For example, the baffle can have only walls that come into physical contact with the bioreactor bag, which have no change in direction in cross-section more kinked than a circular path of a circle having a diameter of at least about one centimeter. Accordingly, a baffle protruding approximately perpendicularly from the container wall and projecting into the receiving space, has a minimum thickness of approximately one centimeter at least at its rounded edge.

The baffle can thus be designed without edges in such a way that it does not have any sharp edges aimed at the receiving space. Additionally or alternatively, the baffle can also be designed without any corner spaces that point away from the receiving space. This can be, for example, corner spaces between a wall of the baffle and the container wall, where air pockets could otherwise form. These corner spaces can also be rounded off in such a way that they do not have a change in direction in cross-section that is more kinked than a circular path of a circle having a diameter of at least approximately one centimeter.

The rounded design of the baffle can reduce air pockets and improve temperature control. As a result, the cultivation of more complicated and/or more intensive cell processes can be made possible.

In particular, the entire receiving space can be designed substantially without edges, i.e. each baffle and each transition between the baffle wall and the container wall has, as described above, no change in direction in cross-section more kinked than a circular path of a circle having a diameter of at least approximately one centimeter.

A third aspect relates to a bioreactor system for receiving a disposable bioreactor bag, which can be for example an embodiment of the bioreactor system according to the first and/or second aspect. The bioreactor system comprises a receiving container having a container wall which defines a receiving space in which the disposable bioreactor bag is received in an operating state of the bioreactor system. A stirring system comprises a stirring shaft, which projects at least partially into the receiving space and is designed and configured to stir a biomedium present in the disposable bioreactor bag in the operating state of the bioreactor system. At least one baffle making the receiving space smaller and differing from the container wall in order to reduce a laminar flow of the biomedium abuts the container wall of the receiving container in such a way that it protrudes from the container wall and projects into the receiving space. The baffle extends in a baffle extension direction along the housing wall of the receiving container. The baffle extension direction is arranged at an angle to the stirring shaft extension direction.

The stirring shaft extension direction extends along the stirring shaft, i.e. the stirring movement of the stirring system takes place by means of a rotation about the stirring shaft extension direction.

The term “angular” here means that the baffle extension direction is not arranged parallel to the stirring shaft extension direction. The baffle extension direction can thus be arranged at an angle to the stirring shaft extension direction, which is at least approximately 1°, preferably at least approximately 2°. The angular arrangement relates to the respectively associated direction vectors, for which reason the actual baffle extension direction does not necessarily have to intersect the stirring shaft extension direction. A projection in two dimensions, e.g. on a vertical plane, can contain one such point of intersection and an angle of intersection, which can be at least about 1°. The directional vectors of the two extension directions preferably form an angle which is no greater than approximately 30°, preferably up to a maximum of approximately 20°, particularly preferably up to a maximum of approximately 10°.

The baffle can extend approximately in a straight line in the baffle extension direction, for example along the container wall from a lower end of the baffle to an upper end of the baffle. As an alternative to this, the baffle can also be arranged in a floor of the receiving container, provided that the angular arrangement is given. However, the baffle is preferably arranged in an approximately vertical side wall of the container wall. The upper end of the baffle can be horizontally offset from the lower end of the baffle, for example offset by at least about 5 cm horizontally. The exact offset depends on the height of the receiving container and can therefore depend on the angle between the extension directions and the working volume.

The baffle can form at least one section of a screw thread along the container wall in the stirring shaft extension direction.

It is essential for this baffle type that the baffle extension direction is not parallel to the rotation vector of the stirring shaft, but rather different therefrom. The baffle acts similarly to a screw thread, whereby the biomedium is not only moved around the stirring shaft by the stirring movement, but can also be raised and/or lowered by the baffle in the stirring shaft extension direction. The inclination of the baffle can, so to speak, screw the biomedium upwards and/or downwards in the receiving space, depending on the direction of stirring. This improves the stirring performance and/or the thorough mixing. The mixing is intensified here, and more intensive bioprocesses can take place in the bioreactor system.

The bioreactor system according to the third aspect can be designed as an embodiment of a bioreactor system according to the first and/or second aspect. Therefore, the descriptions of the corresponding features (e.g. receiving container, receiving space, disposable bioreactor bag, etc.) can apply to all bioreactor systems.

In one embodiment of the bioreactor system according to the third aspect, the baffle is designed as an internal thread of the receiving space along the baffle extension direction. This effect can be intensified in particular by the fact that more than one baffle is formed along the inside of the container wall at an angle to the stirring shaft extension direction and, for example, parallel to one another. As a kind of internal thread, the effect of the vertical mixing is reinforced and/or improved by means of the angular baffle. As a result, the mixing is intensified and/or more effective.

According to one embodiment of the bioreactor system according to the first, second, and/or third aspect, the at least one baffle has a thermal conductivity that is greater than 10 W/(mK). The baffle can be solid, for example. The baffle is thus, for example, metallic and has good thermal conductivity. This also improves the temperature control of the biomedium, because the baffle can easily pass on a temperature control to the biomedium.

A fourth aspect relates to a bioreactor system for receiving a disposable bioreactor bag, which can be an embodiment of a bioreactor system according to the first, second, and/or third aspect. The bioreactor system comprises a receiving container having a container wall which defines a receiving space in which the disposable bioreactor bag is received in an operating state of the bioreactor system. Further, at least one probe window is provided, which allows a view into the inside of the disposable bioreactor bag in the operating state of the bioreactor system. The probe window comprises at least one thermally conductive probe window cover, which is thermally conductively coupled to a cooling system of the bioreactor system.

Probe windows are usually used for coupling probes and/or for illuminating the biomedium. The probe windows can also have ports through which probes can be introduced into the interior of the receiving space. Here, the probe window comprises the probe window cover, which is heat-conducting and is coupled to the cooling system of the bioreactor system. The probe window cover can in particular be movable, for example it can be opened and closed. The probe window cover can have one or more leaf doors, for example. The probe window cover can be metallic, for example, and/or tightly abut the container wall in a closed state covering the probe window. As a result, thermal conduction can be established between the probe window cover and the temperature-controlled container wall via a sufficient coupling surface. Due to the temperature control of the container wall, the temperature of the probe window cover is also controlled, i.e. cooled, for example. Alternatively, the probe window cover itself can be cooled, that is, for example, a temperature control medium can flow through it at least partially.

The cooling system of the bioreactor system can in particular be a cooling of the container wall and/or a cooling of at least one baffle of the bioreactor system.

This type of probe window cover improves the overall cooling of the biomedium, because temperature control is also made possible at the probe window, thus enabling the cultivation of more intensive cell processes.

A fifth aspect relates to a bioreactor system for receiving a disposable bioreactor bag, which can be in particular an embodiment of a bioreactor system according to the first, second, third, and/or fourth aspect. The bioreactor system comprises a receiving container having a container wall which defines a receiving space in which the disposable bioreactor bag is received in an operating state of the bioreactor system. A stirring system projects at least partially into the receiving space and is designed and configured to stir a biomedium present in the disposable bioreactor bag in the operating state of the bioreactor system. The stirring system comprises a stirring shaft, which completely penetrates the receiving space in the operating state of the bioreactor system from a first stirring shaft end to a second stirring shaft end. At least one stirring drive of the stirring system is configured at both the first stirring shaft end and the second stirring shaft end for driving the stirring shaft.

Conventionally, stirring shafts are driven unilaterally, while the other stirring shaft end is freely suspended in the bioreactor. Alternatively, the other stirring shaft end can only be mounted rotatably. The bioreactor system according to the fifth aspect, on the other hand, comprises a stirring shaft which is driven by at least two stirring drives which act on different stirring shaft ends. The first stirring drive is arranged at the first stirring shaft end and the second stirring drive at the second stirring shaft end. Due to the two stirring drives, more stirring power can be applied than with conventional bioreactor systems. This can be, for example, an upper stirring drive and a lower stirring drive. By increasing the total power available, the bioreactor system enables even very viscous cells (such as fungal cells) to be sufficiently mixed so as to enable a corresponding bioprocess.

According to one embodiment, the two stirring drives arranged at the stirring shaft ends can be operated in such a way that they drive the stirring shaft simultaneously and together in the same direction of rotation. In this way, for example, twice the power can be introduced into the biomedium as the stirring power. This improves mixing and allows intensive and/or very viscous biomedia to be cultivated. The two stirring drives are coordinated in such a way that they drive the stirring shaft together in unison, synchronously, and/or at the same speed.

According to one embodiment, the two stirring drives arranged at the stirring shaft ends can be operated in such a way that they drive the stirring shaft in opposite directions of rotation. The stirring drives can be designed in such a way that they drive the stirring shaft either at the same time or at different times. For example, the first stirring drive can be designed only for driving counterclockwise, and the second stirring drive can be designed only for driving clockwise. Depending on the operating state, either the first or the second stirring drive drives the stirring shaft. In particular, however, an operating mode can also be provided in which the two stirring drives simultaneously drive the stirring shaft in opposite directions. For this purpose, the stirring shaft can, for example, be made in several parts, with a first part of the stirring shaft, which is arranged adjacent to the first stirring drive, rotating in a first direction of rotation and a second part of the stirring shaft, which is arranged adjacent to the second stirring drive, rotating in a second opposite direction of rotation. This mixing in different directions of rotation can also lead to a particularly effective and strong mixing of the biomedium and thus makes viscous biomedia accessible for cultivation in the bioreactor.

According to one embodiment, the bioreactor system has a precooling apparatus for precooling the biomedium, which can be fed into the disposable bioreactor bag during a bioprocess. In some bioprocesses, additional (e.g. fresh) biomedium and/or at least components and/or nutrients of the biomedium are introduced into the disposable bioreactor bag during the bioprocess. All of these media introduced during the bioprocess can be precooled by the precooling apparatus and/or can pass through the precooling apparatus. They are therefore introduced into the bioreactor already precooled. This also improves the overall cooling and makes it more effective.

All bioreactor systems described above according to the first to fifth aspects are compatible with one another and relate to the underlying task of enabling a microbial bioprocess and/or phototrophic bioprocess and/or a bioprocess with fungal cells to be carried out. Therefore, all bioreactor systems described above can be designed as embodiments of at least one of the other bioreactor systems. Redundant descriptions are avoided above. The explanations of the corresponding features (such as receiving container, receiving space, disposable bioreactor bag, etc.) can apply to all bioreactor systems that have these features and are therefore also to be understood as a description of these bioreactor systems.

A sixth aspect relates to a method for operating a bioprocess in a disposable bioreactor bag, comprising the following steps
providing a bioreactor system according to the first, second, third, and/or fourth aspect;
inserting the disposable bioreactor bag into the receiving space of the receiving container;
stirring the biomedium present in the disposable bioreactor bag by means of the stirring system; and
reducing a laminar flow of the biomedium by means of the at least one baffle.

The method relates to the operating state and thus the operation of the bioprocess in a bioreactor system according to the first, second, third, and/or fourth aspect. According to these aspects, the description of the bioreactor systems can also relate to the method and vice versa. The baffle is used in order to reduce the laminar flow of the biomedium. The baffle can be cooled and can enable more effective cooling of the biomedium, as described in connection with the first aspect. The baffle can be rounded and can reduce the formation of insulating air pockets, as described in connection with the second aspect. The baffle can be shaped in such a way that it supports and/or improves the mixing in the bioreactor, as described in connection with the third aspect. The cooling can be otherwise enhanced, for example by means of a thermally conductive probe window cover as described in connection with the fourth aspect. Therefore, the method can enable the processing of intensive cell cultures, in particular of microbial processes, phototrophic processes, and/or processes with fungal cells.

A seventh aspect relates to a method for operating a bioprocess in a disposable bioreactor bag, in particular in combination with the method according to the sixth aspect, with the following steps:
providing a bioreactor system according to the fifth aspect;
inserting the disposable bioreactor bag into the receiving space of the receiving container; and
driving the stirring shaft with the two stirring drives in such a way that the biomedium present in the disposable bioreactor bag is stirred.

The stirring shaft can either be driven in such a way that the stirring drives drive the stirring shaft synchronously in the same direction or in opposite directions. The two stirring drives increase the total power that can be introduced into the biomedium and thus enable a more effective mixing of the biomedium, even in the case of viscous biomedia.

According to one embodiment, a precooled biomedium is introduced into the disposable bioreactor bag during the bioprocess. Alternatively or additionally, only components of the biomedium introduced during the bioprocess can be precooled.

According to one embodiment, microbial cells and/or fungal cells are cultivated in the biomedium during the bioprocess. This is made possible by the fact that a particularly effective cooling is used, a particularly high stirring power is provided, and/or both occur. Depending on the process, a correspondingly complex procedure can be used in order to enable the cultivation of even very complex cells.

In the context of this invention, the terms “substantially” and/or “approximately” can be used in such a way that they include a deviation of up to 5% from a numerical value following the term, a deviation of up to 5° from a direction and/or angle following the term.

Unless otherwise specified, terms such as top, bottom, above, below, side, etc. refer to the reference system of the earth in an operating position of the subject-matter of the invention.

The invention is described in further detail below with reference to exemplary embodiments shown in figures. In this case, the same or similar reference numerals can identify the same or similar features of the embodiments. Individual features shown in the figures can be implemented in other exemplary embodiments. The following are shown:

### BRIEF DESCRIPTION OF THE DRAWINGS

FIG. 1 schematically illustrates a perspective view, a bioreactor system for receiving a disposable bioreactor bag;

FIG. 2 schematically illustrates a perspective view, a vertical sectional view through the bioreactor system for receiving a disposable bioreactor bag;

FIG. 3A schematically illustrates a perspective view of a section of an embodiment of a temperature-controlled baffle;

FIG. 3B schematically illustrates a perspective view of an embodiment of a temperature-controlled baffle;

FIG. 3C schematically illustrates a cross-section through an embodiment of a temperature-controlled baffle;

FIG. 4A schematically illustrates a cross-section through an embodiment of a solid baffle;

FIG. 4B schematically illustrates a cross-section through an embodiment of a cavity baffle;

FIG. 5A schematically illustrates a schematic view of an embodiment of a bioreactor system, in whose receiving space a temperature-controlled baffle is arranged;

FIG. 5B schematically illustrates a schematic view of a further embodiment of a bioreactor system, in whose receiving space a temperature-controlled baffle is arranged;

FIG. 6A schematically illustrates a cross-section through a bridge baffle;

FIG. 6B schematically illustrates a cross-section through an elbow baffle;

FIG. 7A schematically illustrates a cross-section through an embodiment of a wave baffle;

FIG. 7B schematically illustrates a cross-section through an embodiment of a double wave baffle;

FIG. 8A schematically illustrates a perspective view of an embodiment of a bioreactor system having a closed probe window cover;

FIG. 8B schematically illustrates a perspective view of an embodiment of a bioreactor system having an open probe window cover;

FIG. 9A schematically illustrates a plurality of views of an embodiment of a receiving container having angular wave baffles; and

FIG. 9B schematically illustrates a plurality of views of an embodiment of a receiving container having angular, rounded bridge baffles.

### DETAILED DESCRIPTION

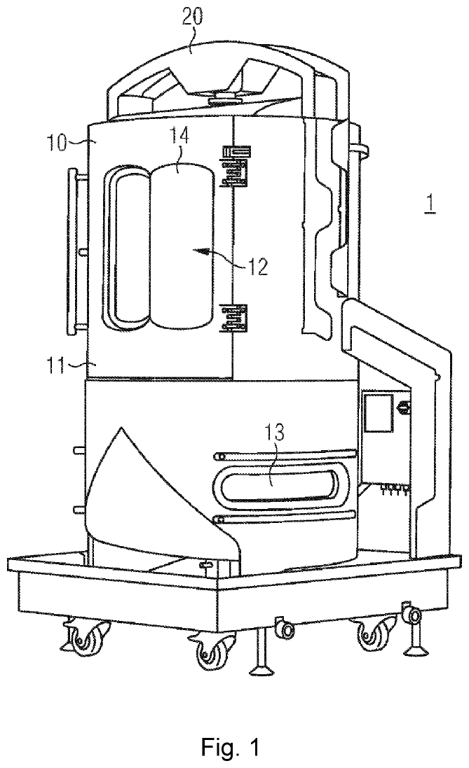

FIG. 1 shows a perspective view of a bioreactor system 1 for receiving a disposable bioreactor bag. A similar bioreactor system is known from the document WO 2016/192824 A1 cited above. This previously known bioreactor system is designed for the cultivation of animal cells in less intensive bioprocesses. There are some structural similarities between the previously known bioreactor system and the embodiment of the bioreactor system 1.

The bioreactor system 1 comprises a receiving container 10 which can substantially have the shape of a vertically arranged cylinder, i.e. the cylinder axis of which can be arranged substantially vertically. The receiving container 10 has a container wall 11 which defines a receiving space 12 into which a disposable bioreactor bag can be inserted, which can contain a biological medium. The receiving space 12 can be designed so as to receive a disposable bioreactor bag with a volume of approximately 5 L to approximately 10,000 L. For example, common disposable bioreactor bags can hold 5 L, 10 L, 50 L, 100 L, 200 L, 500 L, 1,000 L, or 2,000 L of biomedium. The receiving space 12 is preferably designed for the simultaneous cultivation of at least approximately 100 L of biomedium, preferably of at least approximately 500 L, 1,000 L, or in particular even 10,000 L.

The biological medium in the disposable bioreactor bag is stored in the storage space of the storage container 10 for a predeterminable period of time. While the disposable bioreactor bag containing the biological medium is inside the receiving container 10, different reactions with or on the biological medium can occur. In particular, cells can be cultivated in this case.

In order to observe the biological medium, one or more view windows can be formed in the container wall 11, through which it is possible to look from the outside through the container wall 11 into the receiving space 12 of the receiving container 10. This allows the biological medium to be observed.

The bioreactor system 1 can, for example, have at least one bottom view window 13 in the lower third and/or at least one door and/or side view window 14. The bottom view windows 13 can substantially be designed in the form of an elongated oval, whose long oval axis is aligned substantially horizontally along the curved outer cylinder wall of the receiving container 10. The door view window 14 can be configured substantially in the form of an elongated rectangle, whose longer sides are oriented substantially vertically and can be formed approximately in the middle of a single-leaf door in the container wall of the receiving container 10.

The single-leaf door can be rotated about door hinges and is thus openable. If the single-leaf door is open, a door opening is formed in the receiving container 10 at a lateral position, through which access to the interior of the receiving container 10 is made possible. For example, the disposable bioreactor bag can be introduced through the door opening into the receiving space 12 of the receiving container 10 from a lateral direction, that is to say substantially in a horizontal direction of movement.

The bioreactor system 1 can be stored in a rollable manner so that the bioreactor system 1 can be pushed through a room. In addition to rollers, the bioreactor system 1 can have fixing feet at the lower end, which are used in order to fix and correctly align the bioreactor system 1 on uneven floors.

The receiving container 10 can be designed so as to be open at the top. Instead of a cylinder lid, the receiving container 10 can have a stirring opening at its upper end. A part of a stirring system 20 can be formed above the receiving container 10, which is open at the top, in particular a stirring drive of the stirring system 20. A stirring shaft of the stirring system 20 is not explicitly shown and can project through the stirring opening into the receiving space 12 and the disposable bioreactor bag. When operating the stirring shaft, the biomedium can be mixed in the disposable bioreactor bag. The stirring shaft can be designed as a disposable component and can be arranged inside the disposable bioreactor bag. It can be connected to the stirring system 20 via a coupling and/or linkage. The stirring system 20 can be formed centrally over the receiving container 10 and supported by a support bridge abutting an upper edge of the receiving container 10 on opposite side walls of the receiving container 10. The stirring system 20 can comprise other elements, such as another stirring drive present beneath the receiving container 10 for driving a lower end of the stirring shaft. The bioreactor system 1 can also comprise baffles which affect the flow behavior of the biomedium in the receiving space 12, which is mainly caused by the stirring system 20.

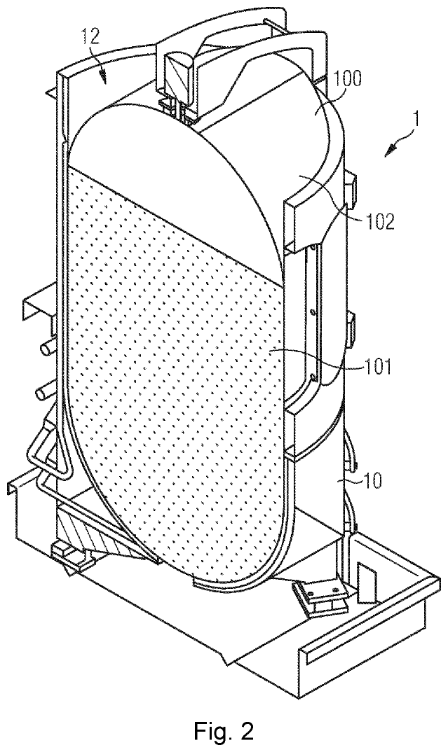

FIG. 2 shows a perspective view of a vertical section through the bioreactor system 1. For example, a disposable bioreactor bag 100 is shown in FIG. 2, more precisely a section through this disposable bioreactor bag 100, which is arranged in the receiving space 12 of the receiving container 10. In the receiving space 12 of the receiving container 10 and at the same time also in the interior of the disposable bioreactor bag 100, there is a biological medium, i.e. a biomedium 101, which can be filled to a predetermined level. The biomedium 101 extends from the bottom of the receiving container 10 up to this filling level and thus fills the entire internal volume of the receiving container 10 up to the filling level, minus the volume of the walls of the disposable bioreactor bag 100, which can be configured very [sic], e.g. from a flexible material (such as plastic) which substantially abuts the inside of the container wall 11.

The disposable bioreactor bag 100 is supported and held in shape by the container wall 11 of the receiving container 10, which extends upwards from the rounded bottom of the receiving container 10 to above the fill level. At least along the upper half, preferably along the upper two-thirds of the receiving container 10, the container wall 11 can substantially extend approximately vertically upwards in the vertical direction.

The container wall 11 can be temperature-controlled by means of a cooling system and/or a cooling apparatus. For this purpose, the container wall 11 can have a cavity, e.g. it can be designed as a double wall, and a temperature control medium (such as e.g. air) can partially flow through it.

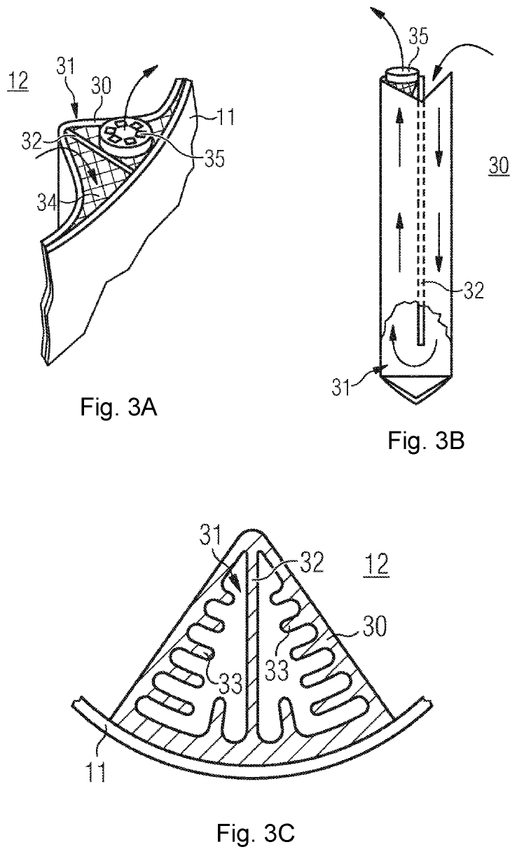

FIG. 3A shows a perspective view of a cross-section through a first baffle 30 of a first baffle type, which is arranged in close contact with the container wall 11 of the receiving container 10 (cf. FIGS. 1 and 2). The baffle 30 of the first baffle type is formed on an inner side of the container wall 11 facing the receiving space 12 in such a way that the baffle 30 is arranged directly adjacent to and/or in physical contact with the container wall 11. The baffle 30 can, for example, extend substantially in a vertical direction from a lower to an upper end of the baffle, e.g. approximately along the entire height along the container wall 11 at least up to the predetermined filling level and/or at least in the approximately vertically arranged part of the container wall 11 (cf. FIGS. 1 and 2).

FIG. 3B shows the full length of the first baffle 30 in a perspective view. The baffle 30 is approximately elongated, and its longitudinal extension axis is approximately vertical.

The cross-section shown in FIG. 3A extends along a plane that is arranged approximately perpendicular to the container wall 11 (here, approximately horizontal) through the baffle 30 and the container wall 11. In the cross-section perpendicular to the container wall 11, the baffle 30 of the first baffle type has an approximately triangular shape. The apex of the triangle faces the receiving space 12 and can, for example, point to a center and/or central axis and/or the cylinder axis of the receiving space 12. A base facing away from this triangle apex can be convex. This base can either be formed directly by the container wall 11, which is slightly curved, for example, or it can be formed separately as a component of the baffle 30 and can be arranged in contact with the container wall 11, which is slightly curved, for example.

The baffle 30 of the first baffle type is at least partially hollow and has a differential temperature control channel 31 in its interior. A channel partition wall 32 is arranged approximately in the middle of the cavity of the baffle 30 in such a way that it mechanically separates the two subchannels of the differential temperature control channel 31 from one another. The channel partition wall 32 can extend from the container wall 11 to the apex of the triangle, which points into the interior of the receiving space 11.

FIG. 3B shows how the channel partition wall 32 can extend almost along the entire length of the baffle 30 of the first baffle type. Only at a (here, the lower) return end of baffle 30 is the channel partition wall 32 interrupted. Otherwise, the channel partition wall 32 separates the cavity of the baffle 30 of the first baffle type approximately evenly into two channel halves, namely a first and a second subchannel, which together form the differential temperature control channel 31.

At a (top) inlet end of baffle 30 of the first baffle type, at which the channel partition wall 32 strictly separates the two subchannels, a temperature control medium, for example a cold one, is introduced into the first sub-channel of the differential temperature control channel 31. This is indicated by the arrows shown in FIGS. 3A and 3B. A liquid or a gas (e.g. air) can be used as the temperature control medium. A filter screen 34 which filters the temperature control medium can be formed at this inlet end, at which the temperature control medium is introduced into the first subchannel. One or more such filter screens 34 can be arranged in the differential temperature control channel 31, in particular at the inlet end.

The temperature control medium introduced in this way runs through the first subchannel of the baffle 30 completely from the inlet end to the opposite return end. At this return end, the channel partition wall 32 is designed so as to be interrupted, and the temperature control medium can flow from the first subchannel of the differential temperature control channel 31 into the second. Along the second subchannel, it flows back from the return end to the outlet end of the baffle 30 of the first baffle type. The outlet end is formed at the same end of the baffle as the inlet end, i.e. at the upper end of the baffle in the exemplary embodiment shown.

The flow of the temperature control medium is indicated by arrows in FIGS. 3A and 3B. The temperature control medium flows through the baffle 30 of the first baffle type twice substantially completely, namely once from the inlet end to the return end and back again to the outlet end, which is arranged adjacent to the inlet end. At least at the outlet end of the differential temperature control channel 31, a ventilator unit 35 can be provided, for example a ventilator wheel, which increases and/or controls and/or regulates the flow rate of the temperature control medium.

The baffle 30 of the first baffle type provides an efficient and effective cooling and/or temperature control of the baffle 30. This enables a temperature control of the biomedium at least on the side surfaces of the baffle 30 of the first baffle type facing the receiving space 12. As a result, the temperature control (and in particular cooling) of the biomedium can be improved during the process.

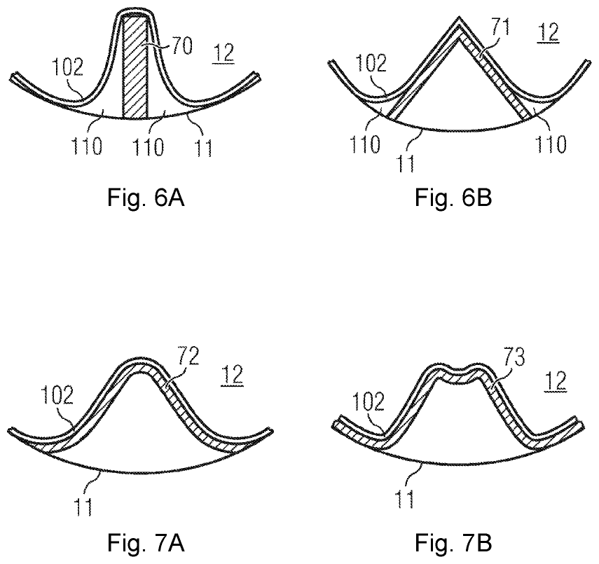

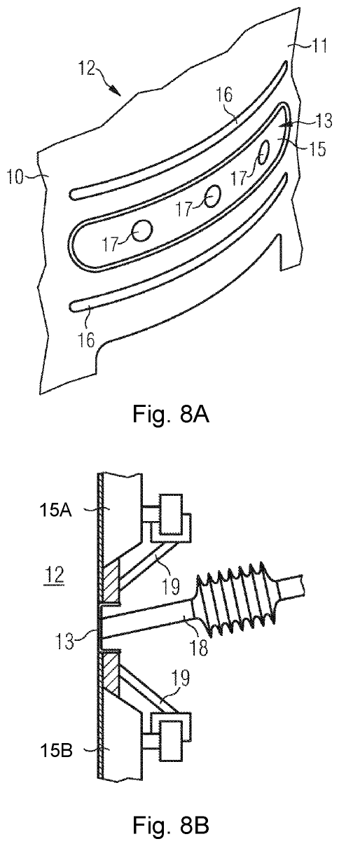

The baffle 30 shown of the first baffle type does not have an independent rear wall, but rather uses the container wall 11 as a rear wall, i.e. as a side wall facing away from the receiving space 1. The transition between the baffle 30 and the container wall 11 is rounded, which will be discussed in further detail below with reference to FIGS. 6 and 7.

FIG. 3C shows a cross-section through a further embodiment of a baffle 30 of the first baffle type approximately perpendicular to the container wall 11. This is a baffle 30 which, by contrast to the baffle 30 shown in FIGS. 3A and 3B, has its own rear wall which tightly abuts the container wall 11. Just like the baffle 30 shown in FIGS. 3A and 3B, the baffle 30 shown in FIG. 3C also has a differential temperature control channel 31, by means of which the baffle 30 can be temperature-controlled.

The baffle 30 of the first baffle type not only has the channel partition wall 32 on the inside, but also one or more cooling web(s) 33. The cooling webs 33 can be arranged on an inner wall of the baffle 30, whose outer wall facing away from the inner wall is in direct physical contact with the disposable bioreactor bag in the operating position. The baffle walls of the baffle 30 thus represent a direct delimitation of the receiving space 12 of the bioreactor system 1. The cooling web 33 can be solid and projects approximately perpendicularly from the inner wall of the baffle 30 into the cavity and/or the subchannels of the baffle.

In general, the baffle 30 of the first baffle type, like the baffle of a second baffle type, is designed as an obstacle that narrows and/or delimits the receiving space 12 and serves to reduce a laminar flow in the receiving space 12. The baffles of the first baffle type differ from the baffles of the second baffle type, described in further detail below, in that they abut the container wall 11 at least in sections or even completely, while the baffles of the second baffle type run through the interior of the receiving space 12, e.g. approximately parallel and spaced apart from the container wall 11.

The cooling webs 33 of the baffle 30 of the first baffle type shown in FIG. 3C is or are preferably made of a thermally conductive material, as are the other baffle walls, for example. In particular, metallic materials such as stainless steel are particularly suitable. The cooling webs 33 can improve a heat exchange between the temperature control medium and the walls of the baffle 30 and can thus enhance the cooling effect of the baffle 30 of the first baffle type.

The baffle 30 of the first baffle type shown in FIGS. 3A and 3B can also have cooling webs 33, i.e. just like the baffle 30 of the first baffle type shown in FIG. 3C.

As an alternative to the differential temperature control channel 41, the baffle 30 can also have a single-channel temperature control channel, with which it is penetrated only once and can be temperature controlled.

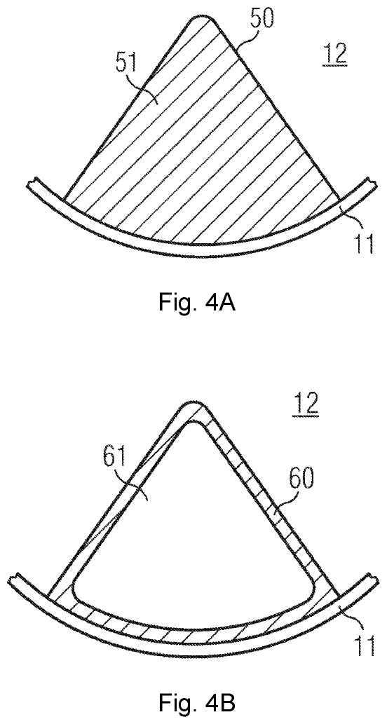

FIG. 4A shows a cross-section approximately perpendicular to the container wall 11 through an embodiment of a solid baffle 50 of the first baffle type. The solid baffle 50 has an approximately triangular cross-sectional shape, similar to the baffles 30 shown in FIGS. 3A-3C. By contrast to this, however, the solid baffle 50 is designed with a solid baffle body 51. The solid baffle body 51 is formed entirely of a material having good thermal conductivity, for example a metal such as aluminum.

Two outer walls of the solid baffle 50, which is triangular in cross-section, face the receiving space 12, while a third outer wall, as the rear wall, is in close thermal contact with the container wall 11 without any gaps. As a result, the rear wall of the overall thermally conductive solid baffle 50 is closely and thermally conductively coupled to the container wall 11, so that it is also temperature-controlled by a temperature control and/or cooling system of the container wall 11. As described above, the container wall 11 can be temperature-controlled. For example, the container wall 11 can be designed as a double wall, in which a temperature control medium and/or coolant circulates for temperature control and/or cooling. Due to the close thermal coupling, the solid baffle 50 benefits from the temperature control of the container wall and can pass this temperature control and/or cooling on to the biomedium present in the receiving space 12.

FIG. 4B shows an embodiment of a cavity baffle 60 of the first baffle type. The cavity baffle 60 can also be approximately triangular in the cross-section shown, approximately perpendicular to the container wall. In this case, two side walls face the receiving space 12, and a third rear side is designed without a gap and/or so that it tightly abuts the container wall 11. As a result, the cavity baffle 60 is also well coupled to the temperature control and/or cooling system of the container wall 11 and can pass this temperature control and/or cooling on to the biomedium present in the receiving space 12.

The baffles 30, 50, 60 of the first baffle types need not necessarily be triangular in cross-section, but can have other shapes, such as a wavy shape and/or a square shape. However, they can all have a rear side which tightly abuts the container wall 11 (similar to what is shown in FIGS. 3C, 4A, 4B) or is even formed by it (similar to what is shown in FIG. 3A).

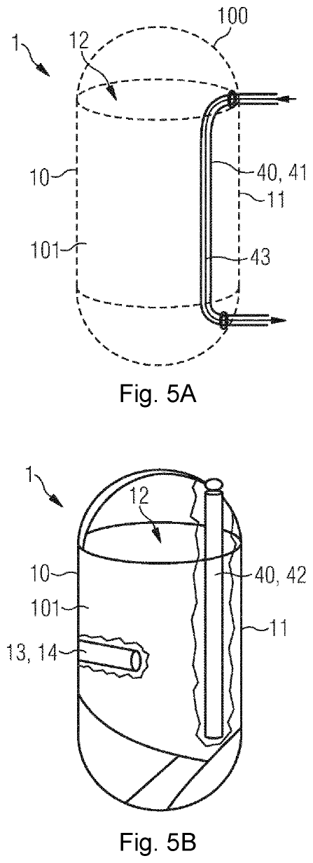

FIG. 5A shows a schematic view of an embodiment of a bioreactor system 1, in whose receiving space 12 a baffle 40 of a second baffle type is arranged. The baffles of the second baffle type differ from those of the first baffle type, in that they run and/or project into and/or through the receiving space 12 spaced apart from the container wall 11 at least in sections. By contrast to this, the baffles of the first baffle type are arranged so as to tightly abut an inside of the container wall 11.

The baffle 40 of the second baffle type shown, on the other hand, is arranged spaced apart from the container wall 11 for the most part in such a way that the biomedium 101 substantially flows around it from all radial and/or horizontal directions. The baffle 40 of the second baffle type shown in FIG. 4A is a baffle 41 of the second baffle type fastened on both sides, which leads through the receiving space 12 spaced apart from the container wall 11 for the most part. The elongated baffle 41 can be fastened to the container wall 11 at both of its baffle ends and can even break through it. The baffle 41 can completely penetrate the receiving space 12 from its upper baffle end to its lower baffle end.

A temperature control medium can flow through the baffle 41 fastened on both sides along its entire length, which is indicated by arrows in FIG. 5A. For this purpose, the baffle 41 has a temperature control channel 43 which extends as a cavity along the extension between the baffle ends of the baffle 41 fastened on both sides. The temperature control channel 43 can extend at least from a first fastening end of the baffle 41 to a second fastening end of the baffle 41 and optionally also beyond, e.g. from a temperature control medium source to a temperature control medium outlet.

A liquid and/or gaseous temperature control medium can flow through the temperature control channel 43, as a result of which the baffle 41 fastened on both sides is temperature controlled and provides a temperature sink in the middle of the biomedium 101.

The baffle 41 fastened on both sides can be designed as a pressurized, welded hose. The baffle 41 can be designed as an integral part of the disposable bioreactor bag 100. During assembly, the temperature control channel 43 can be connected to one or more channels of a temperature control medium at the fastening ends.

The temperature control in the midst of the biomedium 101 enables an effective and efficient temperature control of the biomedium. At least one wall of the baffle 41 can be transparent and can be made of a transparent plastic, for example. A luminous and/or fluorescent liquid can then be used as a temperature control medium in photobioreactors. This can make the bioreactor system 1 usable for intensive phototrophic bioprocesses.

The baffle 41 does not necessarily have to be pressurized from the outset, but can merely be designed as an initially slack hose tunnel through the interior of the disposable bioreactor bag 100. By pressurizing the at least one baffle 41 in a state of having been inserted into the receiving container 10, the disposable bioreactor can erect itself in such a way that it can be easily connected to ports of the receiving container 10 even before it is filled with the biomedium. Thus, the free baffles 41 fastened on both sides can simplify and/or facilitate assembly and/or construction of the disposable bioreactor bag 100 in the bioreactor system 1.

As an alternative to the single-channel temperature control channel 43, the baffle 41 can be penetrated by a differential temperature control channel, similar to the baffles 30 shown in FIGS. 3A-3C.

FIG. 5B shows a perspective view of a further exemplary embodiment of a bioreactor system 1 having a further baffle 40 of the second baffle type. This baffle 40 is a unilaterally fastened baffle 42. This unilaterally fastened baffle 42 can, for example, hang vertically downwards into the receiving space 12 from an upper end of the receiving space 12. Alternatively, the unilaterally fastened baffle can also project into the interior of the receiving space 12 from a different direction, in particular from the side or from below. However, the unilaterally fastened baffle 42 is preferably aligned in such a way that it extends substantially parallel to an extension direction (not shown in the figures) of a stirring shaft of the stirring system of the bioreactor system 1. This can prevent the baffle 42 and the stirring shaft from interfering too much. Furthermore, the unilaterally fastened baffle 42 can then be configured as long as possible.

This extension direction approximately parallel to the stirring shaft is also advantageous for the baffle 41 shown in FIG. 5A, which is fastened bilaterally.

A temperature control medium can also flow through the unilaterally fastened baffle 42. For example, it can have an opposing temperature control channel on the inside, similar to the baffle 30, which is shown in FIGS. 3A and 3B. Alternatively, the baffle 42 can also be solid, for example, similar to the solid baffle 50 shown in FIG. 4A. In the disposable bioreactor bag 100, the baffle 42 can be designed merely as a foil insert into which a cooling finger of the bioreactor system 1 can be introduced. The cooling finger can, for example, be solid or designed as a hollow body, i.e. similar to the baffles 50 or 60 shown in FIGS. 4A and 4B. Furthermore, an opposing temperature control channel can also be arranged in the cooling finger, similar to the baffle 30 of the first baffle type (cf. FIGS. 3A to 3C).

The unilaterally fastened baffle 42 has a free end, which is arranged facing away from the fastened end and projects into the interior of the receiving space 12. The biomedium 101 can completely flow around this free end.

The baffles 41 and 42 of the second baffle type improve the temperature control of the biomedium 101 by providing a heat sink directly inside the biomedium 101, for example. This can enable the processing of intensive cell cultures, which for example require a stronger cooling capacity than conventional animal bioprocesses. The bioreactor systems 1 shown in FIGS. 5A and 5B are to be understood to be examples. A plurality of baffles 40, 41, and/or 42 can be arranged therein in order to improve the temperature control.

FIGS. 6A and 6B each show a cross-section approximately perpendicular to the container wall 11 through a bridge baffle 70 (FIG. 6A) and an angular baffle 71 (FIG. 6B). In the cross-section shown, the bridge baffle 70 is designed as a substantially rectangular bridge, which projects approximately perpendicularly from the container wall 11 into the interior of the receiving space 12. In the cross-section shown, the angular baffle 71 is designed as an angle with legs of approximately the same length, wherein the vertex of the angle points into the interior of the receiving space 12.

The baffles 70, 71 shown in FIGS. 6A and 6B have the disadvantage that they have relatively sharp edges and/or form angular transitions with the container wall 11. When inserting the disposable bioreactor bag 100, the bag wall 102 abuts the baffles 70 and 71 so unfavorably that air pockets 110 can form between the bag wall 102 on the one hand and the bridge baffle 70 or angular baffle 71 on the other hand and possibly the container wall 11.

These air pockets 110 can occur in particular at a transition between the container wall and the baffle wall of baffle 70 or 71. The baffle walls can protrude from the container wall 11 at a clearly defined angle of, for example, about 30 to about 120 degrees. The flexible bag wall 102 cannot tightly abut this transition, for which reason the air pockets 110 are created. The air pockets 110 can develop an insulating effect, which impedes and/or degrades the cooling of the biomedium 101, because they act as an insulation between the temperature-controlled container wall 11 and the biomedium 101.

FIGS. 7A and 7B show a cross-section approximately perpendicular to the container wall 11 through a wave baffle 72 and a double wave baffle 73. The wave baffles 72 and the double wave baffles 73 are much better suited for the effective cooling of the biomedium 101 than the bridge baffles 70 and the angular baffles 71 shown in FIGS. 6A and 6B. For example, the wave baffles 72 and the double wave baffles 73 are each configured as a rounded baffle of the first baffle type. The rounded baffles 72, 73 are designed so that they are free of sharp edges so that the bag wall 102 can tightly abut the baffles 72, 73 and also the container wall 11 in the operating state. As a result, insulating air pockets 110 between the bag wall 102 and the temperature-controlled container wall 11 and the baffles 72, 73 are reduced. In addition, a contact region between the container wall 11 and the baffles 72, 73 on the one hand and the bag wall 102 and thus the biomedium on the other hand can be increased. Furthermore, stress on the bag wall 102 of the disposable bioreactor bag 100 can be reduced as a result. These effects can be achieved by the outer surfaces of the baffle walls of the baffles 72, 73 sliding gently in cross-section, which allows the disposable bioreactor bag 100 to tightly abut the boundaries of the receiving space 12, in particular on and adjacent to the baffles 72, 73.

In the cross-section shown, the wave baffle 72 is designed as an approximately single-humped wave with a rounded wave crest and additionally rounded flanks, which nestle against the transition to the container wall 11 without any edges. Both the wave crest and the wave troughs adjacent to the container wall 11 form a curved shape in cross-section with a curve diameter that is preferably at least approximately 1 cm.

The same applies to the double wave baffle 73, which, in the cross-section shown, is designed similarly to the wave baffle 72 but, in contrast thereto, has a double wave as a double hump. This double wave is also rounded and additionally has rounded flanks, which nestle against the transition to the container wall 11 without any edges. Both the double wave crest and the wave troughs adjacent to the container wall 11 have a curved cross-section with a curve diameter that is preferably at least approximately 1 cm.

In an alternative embodiment, the wave baffles can have additional wave crests, e.g. as a triple or quadruple wave baffle. The crests and/or troughs of the waves can be of different heights.

Both the wave baffle 72 and the double wave baffle 73 can be configured as a cavity baffle (similar to the cavity baffle 60 shown in FIG. 4B), as a solid baffle (similar to the solid baffle 50 shown in FIG. 4A), and/or as a temperature-controlled baffle with a simple or differential temperature control channel, i.e. similar to the baffles shown in FIG. 3 or 5.

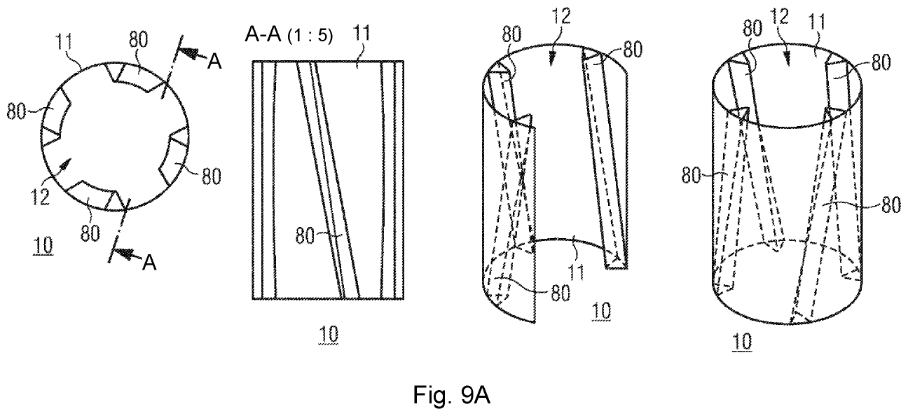

FIG. 8A shows a perspective view of a bioreactor system with a bottom view window 13 which is covered by a view window cover 15. As shown in FIG. 1, the bottom view window 13 can be formed in the container wall 11 in a lower region of the receiving container 10. A probe holder 16 in the form of a handlebar can be arranged above and/or below the bottom view window 13. Probes can be fastened to the probe holder 16 and can be arranged on the bottom view window 13. Such probes can, for example, reach into the interior of the receiving space 12 and take measurements there.

Conventional bottom view windows 13 are not temperature-controlled, but are made of glass, for example. In the embodiment shown in FIG. 8A, the bottom view window 13 is covered by the view window cover 15. The view window cover 15 can be thermally conductive and/or thermally conductively coupled to a cooling system of the container wall 1. For this purpose, the view window cover 15 can be formed, for example, from a metal such as aluminum. The view window cover 17 can include one or more probe inlets 17 through which probes can be fastened to or through the bottom view window 13.

A similar view window cover can also be arranged on a side view window 14 of the bioreactor system 1 (cf. FIG. 1).

FIG. 8B shows in a perspective view that the view window cover 15 can be opened. More specifically, the view window cover 15 has a first cover flap 15A and a second cover flap 15B. These can be folded away from the bottom view window 13 in such a way that they release the bottom view window 13. For this purpose, at least one hinge 19, preferably one hinge 19 for each cover flap 15A, 15B, can be provided, which serves to open and/or close the cover flaps 15A and/or 15B.

The thermally conductive view window cover(s) 15/15A/15B enable a thermal coupling of the region of the view window 13, 14 to the temperature control of the container wall 11, e.g. its temperature control by means of a temperature control medium in the double wall. This makes it possible to cool the biomedium 101 on the surface occupied by the view windows 13, 14, as well. As a result, the cooling is improved overall, and more intensive cell culture processes can be made possible.

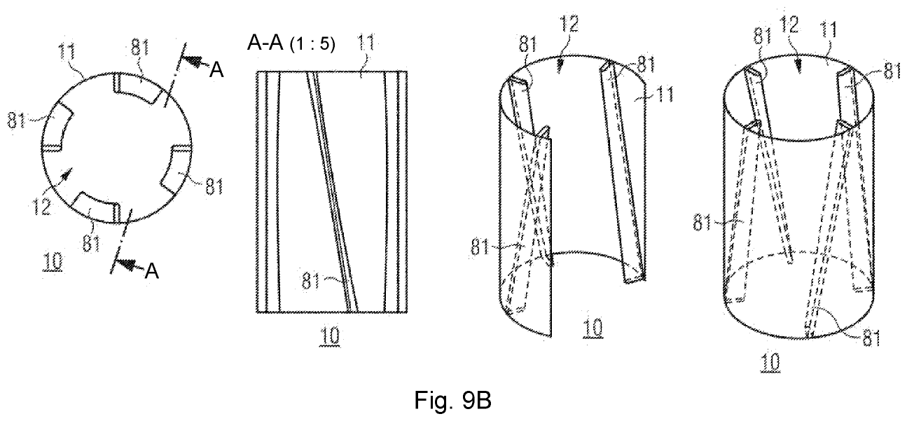

FIG. 9A shows several views of an embodiment of a receiving container 10 having angular wave baffles 80. A view from above into the receiving space 12 of the receiving container 10 is shown on the far left in FIG. 9A. Here, a marking of a section along a vertical plane through the receiving container 10 is shown, which is shown to the right as a sectional view. The third view from the left shows a partially open perspective view of the receiving container 10, and a closed perspective view of the receiving container 10 on the far right.

A plurality of wave baffles 80 of the first baffle type are arranged in the receiving container 10, which are arranged in close contact with or adjacent to the container wall 11 of the receiving container 10. In the exemplary embodiment shown, the receiving container 10 has exactly four such angular wave baffles 80. The cross-section through the wave baffle 80 can be formed approximately like that of the wave baffle 72 shown in FIG. 7A. Alternatively, the angular wave baffle 80 can be solid (like the solid baffle 50 shown in FIG. 4A) or hollow, such as the cavity baffle 60 shown in FIG. 4B. The angular wave baffle 80 can also be a temperature control channel, e.g. similar to the baffles 30 shown in FIGS. 3A to 3C.

The angular wave baffle 80 extends from a lower end to an upper end along the container wall 11 in a roughly vertical direction. However, the upper end is offset horizontally to the lower end. Thus, the angular wave baffle 80 does not extend exactly in the vertical direction, but rather at an angle to the vertical along the inside of the container wall 11. The wave baffles 80 form a type of internal screw thread in the receiving space 12.

In particular, taken together with all of the angular wave baffles 80, a plurality of barriers are thus formed in the receiving space 12, which are all arranged approximately in the same direction and offset approximately parallel to one another at an angle to the vertical. This barrier, which is similar to an internal thread, causes the biomedium 101 to be “screwed” either upwards or downwards (depending on the direction of stirring) during the rotational mixing of the biomedium 101 through the barriers formed by the angular wave baffles 80. Due to the angular arrangement of the wave baffles 80, a vertical movement component is generated when the biomedium 101 is mixed.

The angular wave baffles 80 extend angularly to a stirring shaft (not shown in the figures) and its axis of rotation. The angle between the extension direction of the angular wave baffles 80 and the stirring shaft extension direction can be at least approximately 5° in order to generate a sufficient vertical stirring movement.

FIG. 9B shows, similar to FIG. 9A, an embodiment of a receiving container 10 with angular bridge baffles 81. Here, the angular baffles are designed as angular, rounded bridge baffles 81. They have a similar shape to the bridge baffles 70 shown in FIG. 6A, but are rounded off at least on their edge projecting into the receiving space 12 in such a way that they do not form any sharp edges there. This shape of the angular bridge baffles 81 also enables—similar to the angular wave baffles 80 shown in FIG. 9A—an additional directional and/or mixing component of the biomedium 101 in the verticals and thereby intensifies the mixing.

The embodiments of the receiving containers 10 with the angular baffles 80, 81 shown in FIGS. 9A and 9B improve and/or intensify the mixing. This can enable the realization of bioreactors for cultivating relatively viscous cells.

The baffles shown in FIGS. 3A-3C, 4A, 4B, 5A, 5B, 7A, and 7B improve the cooling of the biomedium 101, because they enable the baffles themselves to be temperature-controlled and/or allow temperature drops inside the bioreactor. This can enable the realization of bioreactors for cultivating cells that require intensive cooling.

The probe window cover 15 shown in FIGS. 5A and 5B also improves the cooling of the biomedium 101, because it increases the available temperature control surface. This can enable the realization of bioreactors for cultivating cells that require intensive cooling.

The measures outlined above can be combined with one another in order to further improve the cooling and/or mixing through combination.

According to one embodiment, a stirring system is used which is operated at a stirrer peripheral speed of up to about 6.0 m/s. As a result, the oxygenation and the mixing of the biomedium 101 can be improved.

According to one embodiment, a stirring system with a power input of up to about 11 kW/m³ is used. This enables, for example, the high stirrer peripheral speed of approximately 6 m/s.

According to one embodiment, a gassing rate of up to about 3.0 vvm (abbreviation for “vessel volume per minute”) is used. The oxygenation into the biomedium 101 can also be improved in this way. Furthermore, as a side effect of this, so to speak, the mixing effect can also be improved.

According to one embodiment, the bioreactor system 1 is optimized in order to use a La value of up to 1,000 per hour. This can be achieved as a consequence of the high stirrer peripheral speed and/or the high gassing rate.

According to one embodiment, a heating and/or cooling rate, i.e. generally a temperature control rate, of up to 90 watts per liter of the biomedium is used.

According to one embodiment, no plastic parts and/or only as few plastic parts as possible are used in the stirring drive of the stirring system. A stirring shaft made of stainless steel and/or steel can preferably be used in order to enable a high transmission of force. The stirrer itself can also be made of metal in order to enable a high transmission of force into the biomedium.

According to one embodiment, a stirrer with a geometry suitable for a power input is arranged on the stirring shaft of the stirring system. In this case, for example, stirrer geometries can be used which are known under the name Smith and, if applicable, variants. The stirrer can be designed as a hydrofoil and/or as a closed Smith stirrer. Alternatively, an elephant-ear geometry can be used, or an impeller bulletin, which is a subtype of the Smith stirrer. The stirrer can be made of stainless steel in order to be able to mix highly viscous cells (e.g. fungal cells).

According to one embodiment, a flow breaker is arranged on the stirrer, e.g. a circular disk, which surrounds the tips of the stirrer and thus achieves an improved mixing effect.

According to one embodiment, the receiving container 10 has a height at least three times its diameter. The receiving container 1 is of a substantially cylindrical design, as also shown in FIGS. 1, 2, 5A, 5B, 9A, and 9B. This relatively tall and slim design of the receiving container 10 increases the cooling surface, because a larger amount of the biomedium 101 is present on the temperature-controlled container wall 11 per volume. Furthermore, the gas dwell time can also be increased as a result.

According to one embodiment, both a gas flowing in above the biomedium 101 and/or a liquid feed for the biomedium 101 can be precooled in order to improve the overall cooling capacity.

According to one embodiment, the receiving container 10 can be designed without a door and/or without a view window. A camera for observing the biomedium 101 can be placed inside the bag holder and/or even inside the disposable bioreactor bag 100. This also increases the overall effective region of the temperature-controlled container wall 11.

According to one embodiment, the flow rates of a temperature control medium in the temperature-controlled double sheath of the container wall 11 are optimized, in particular increased, for the cooling capacity to be achieved.

Furthermore, in order to achieve a stronger stirring performance, the strength of a magnetic disk of the stirring drive can be optimized, as well as the strength, number, length, and/or quality of the magnets for coupling the stirring drive to the inside of the disposable bioreactor bag 100.

The power of the stirring system can be transferred to the interior of the disposable bioreactor bag 100 by means of a radial magnetic coupling with fine scaling. This allows the torque to be increased.

Long and strong magnets and/or more magnets overall can be used in order to improve the coupling between the stirring drive and the stirring shaft present inside the disposable bioreactor 100. In this case, current-induced magnetization can be used in order to improve the magnetic coupling.

These and other optimizations can be made in order to optimize the bioreactor system for use in bioprocesses on intensive cell cultures.

Overall, the invention provides a bioreactor system 1 and a method for operating it, in which the mixing is improved, the temperature control and/or cooling capacity is increased, and thus the cultivation of cell cultures that were previously inaccessible to the bioprocess is made possible.

### LIST OF REFERENCE NUMERALS

1 Bioreactor system
10 Receiving container
11 Container wall
12 Receiving space
13 Bottom view window
14 Side view window
15 View window cover
15A First cover flap
15B Second cover flap
16 Probe holder
17 Probe aperture
18 Probe
19 Hinge
20 Stirring system
30 Baffle of a first baffle type
31 Differential temperature control channel
32 Channel partition wall
33 Cooling bridge
34 Filter screen
35 Ventilator unit
40 Baffle of a second baffle type
41 Free bilaterally fastened baffle
42 Free unilaterally fastened baffle
43 Temperature control channel
50 Solid baffle
51 Solid baffle body
60 Cavity baffle
61 Cavity
70 Bridge baffle
71 Elbow baffle
72 Wave baffle
73 Double wave baffle
80 Angular wave baffle
81 Angular, rounded bridge baffle
100 Disposable bioreactor bag
101 Biomedium
102 Bag wall
110 Air cushion

1. A bioreactor system for receiving a disposable bioreactor bag, the bioreactor system comprising:
a receiving container having a container wall which defines a receiving space in which the disposable bioreactor bag is received in an operating state of the bioreactor system;
a stirring system which projects at least partially into the receiving space and is designed and configured to stir a biomedium present in the disposable bioreactor bag in the operating state of the bioreactor system; and
at least one baffle, which makes the receiving space smaller and differs from the container wall, serving to reduce a laminar flow of the biomedium;
wherein a temperature control medium flows through at least part of the at least one baffle, said temperature control medium controlling the temperature of the baffle.

2. The bioreactor system according to claim 1, wherein the at least one baffle comprises at least one baffle of a first baffle type, which rests against the container wall of the receiving container in such a way that it protrudes from the container wall and projects into the receiving space.

3. The bioreactor system according to claim 1, wherein the at least one baffle comprises at least one baffle of a second baffle type, which extends at least along a portion spaced apart from the container wall of the receiving container through the receiving space.

4. The bioreactor system according to claim 1, wherein the baffle comprises a differential temperature control channel, through which the temperature control medium flows through the baffle in two opposite directions.

5. The bioreactor system according to claim 1, wherein, within the baffle, at least one cooling bridge is arranged on at least one baffle wall, which, in the operating state of the bioreactor system, is abutted by a bag wall of the disposable bioreactor bag.

6. The bioreactor system according to claim 1, wherein the baffle penetrates the receiving space approximately completely along an approximately vertical direction.

7. The bioreactor system according to claim 1, wherein the baffle is configured so as to project from one end of the receiving space into the receiving space.

8. A bioreactor system for receiving a disposable bioreactor bag, the bioreactor system comprising:
a receiving container having a container wall which defines a receiving space in which the disposable bioreactor bag is received in an operating state of the bioreactor system;
a stirring system which projects at least partially into the receiving space and is designed and configured to stir a biomedium present in the disposable bioreactor bag in the operating state of the bioreactor system; and
at least one baffle, which makes the receiving space smaller and differs from the container wall, serving to reduce a laminar flow of the biomedium, which abuts the container wall of the receiving container in such a way that it protrudes from the container wall and projects into the receiving space;
wherein the baffle is configured so as to be rounded, such that a wall of the baffle and/or at least a transition from the container wall of the receiving container to the wall of the baffle abutting the former, which wall and/or transition is abutted by disposable bioreactor bag in the operating state, is configured so as to be substantially edgeless.

9. A bioreactor system for receiving a disposable bioreactor bag, the bioreactor system comprising:
a receiving container having a container wall which defines a receiving space in which the disposable bioreactor bag is received in an operating state of the bioreactor system;
a stirring system with a stirring shaft, which projects at least partially into the receiving space and is designed and configured to stir a biomedium present in the disposable bioreactor bag in the operating state of the bioreactor system; and
at least one baffle, which makes the receiving space smaller and differs from the container wall, serving to reduce a laminar flow of the biomedium, which abuts the container wall of the receiving container in such a way that it protrudes from the container wall and projects into the receiving space;
wherein the baffle extends in a baffle extension direction along the housing wall of the receiving container and the baffle extension direction is arranged at an angle to a stirring shaft extension direction of the stirring shaft.

10. The bioreactor system according to claim 9, wherein the baffle is configured as an internal thread of the receiving space along the baffle extension direction.

11. The bioreactor system according to claim 9, wherein the at least one baffle is formed from a material having a thermal conductivity that is greater than 10 W/mK and/or is solid in form.

12. The bioreactor system according to claim 1 comprising:
at least one probe window, which allows a view into the inside of the disposable bioreactor bag in the operating state of the bioreactor system;
wherein the probe window comprises at least one thermally conductive probe window cover, which is thermally conductively coupled to a cooling system of the bioreactor system.

13. The bioreactor system according to claim 1 comprising:
a stirring system which projects at least partially into the receiving space and is designed and configured to stir a biomedium present in the disposable bioreactor bag in the operating state of the bioreactor system;
wherein
the stirring system comprises a stirring shaft, which completely penetrates the receiving space in the operating state of the bioreactor system from a first stirring shaft end to a second stirring shaft end; and
at least one stirring drive of the stirring system is configured at both the first stirring shaft end and the second stirring shaft end for driving the stirring shaft.

14. The bioreactor system according to claim 13, wherein the two stirring drives arranged at the stirring shaft ends can be operated in such a way that they drive the stirring shaft simultaneously and together in the same direction of rotation.

15. The bioreactor system according to claim 13, wherein the two stirring drives arranged at the stirring shaft ends can be operated in such a way that they drive the stirring shaft in opposite directions of rotation.

16. The bioreactor system according to claim 13, having a precooling apparatus for precooling a biomedium and/or a component of the biomedium, which can be conducted into the disposable bioreactor bag during a bioprocess.

17-20. (canceled)
